(19) United States
(12) Patent Application Publication
(10) Pub. No.: US 2023/0285641 A1
(43) Pub. Date: Sep. 14, 2023

(54) THROMBORESISTANT COATINGS, COATED DEVICES, AND METHODS

(71) Applicant: Surmodics Coatings, LLC, Eden Prairie, MN (US)

(72) Inventors: Koren Fitzmorris, Bloomington, MN (US); Syed Hossainy, Hayward, CA (US); Sean M. Stucke, Farmington, MN (US); Michael Militello, Eden Prairie, MN (US); Toni M. Heyer, St. Louis Park, MN (US)

(21) Appl. No.: 18/119,693

(22) Filed: Mar. 9, 2023

Related U.S. Application Data
(60) Provisional application No. 63/318,275, filed on Mar. 9, 2022.

(51) Int. Cl. A61L 33/00; B08B 17/02

(52) U.S. Cl. CPC A61L 33/0005; B08B 17/02; A61L 33/0011; A61L 2300/42; A61L 2400/10

(57) ABSTRACT

Embodiments herein relate to thromboresistant coatings, coated devices, and related methods. In an embodiment, a thromboresistant implantable, partially implantable, or wearable medical device is included having a substrate, a non-fouling basecoat layer, and a lubricious topcoat layer. The non-fouling basecoat layer can include a hydrophilic component and a hydrophobic component. The non-fouling basecoat layer is disposed over the substrate. The lubricious topcoat layer can include a photo-reactive polyvinylpyrrolidone compound and a cross-linking agent. The lubricious topcoat layer can be disposed over the non-fouling basecoat layer. Other embodiments are also included herein.

This application claims the benefit of U.S. Provisional Application No. 63/318,275, filed Mar. 9, 2022, the content of which is herein incorporated by reference in its entirety.

### FIELD

Embodiments herein relate to coatings for medical devices. More specifically, embodiments herein relate to thromboresistant coatings, coated devices, and related methods.

### BACKGROUND

Medical devices include, amongst others, those that are chronically implanted, devices that are transitorily implanted, those that not implanted at all but in contact with tissue and/or bodily fluids, and wearable devices amongst others. Many types of medical devices are enhanced by coatings that can provide various useful properties to the surfaces of medical device.

Biofouling is a significant problem with medical devices that may contact tissue and/or bodily fluids. The biofouling process starts with protein binding to the surface, followed by cells attaching to the proteins on the surface. Biofouling can lead to device malfunctioning, reduced sensitivity of sensors, foreign body reactions, and infections.

### SUMMARY

Embodiments herein relate to thromboresistant coatings, coated devices, and related methods. In a first aspect, a thromboresistant implantable, partially implantable, or wearable medical device can be included having a substrate and a non-fouling basecoat layer. The non-fouling basecoat layer can include a hydrophilic component and a hydrophobic component and can be disposed over the substrate. The medical device can also include a lubricious topcoat layer including a photo-reactive polyvinylpyrrolidone compound and a cross-linking agent. The lubricious topcoat layer can be disposed over the non-fouling basecoat layer.

In a second aspect, in addition to one or more of the preceding or following aspects, or in the alternative to some aspects, the hydrophobic component can include a poly(butyl methacrylate).

In a third aspect, in addition to one or more of the preceding or following aspects, or in the alternative to some aspects, the hydrophobic component can include a poly(n-butyl methacrylate).

In a fourth aspect, in addition to one or more of the preceding or following aspects, or in the alternative to some aspects, the hydrophilic component can include a polyvinylpyrrolidone polymer.

In a fifth aspect, in addition to one or more of the preceding or following aspects, or in the alternative to some aspects, the polyvinylpyrrolidone polymer can include a cross-linked polyvinylpyrrolidone.

In a sixth aspect, in addition to one or more of the preceding or following aspects, or in the alternative to some aspects, the hydrophilic component can further include an acrylamide polymer, wherein the polyvinylpyrrolidone polymer and the acrylamide polymer can be cross-linked.

In a seventh aspect, in addition to one or more of the preceding or following aspects, or in the alternative to some aspects, a ratio of the hydrophobic component to the hydrophilic component in the non-fouling basecoat layer can be from about 1:1 to 8:1 by weight.

In an eighth aspect, in addition to one or more of the preceding or following aspects, or in the alternative to some aspects, a ratio of the hydrophobic component to the hydrophilic component in the non-fouling basecoat layer can be from about 2.5:1 to 3.5:1 by weight.

In a ninth aspect, in addition to one or more of the preceding or following aspects, or in the alternative to some aspects, the non-fouling basecoat layer can further include a heparin compound.

In a tenth aspect, in addition to one or more of the preceding or following aspects, or in the alternative to some aspects, the heparin compound can include a photoreactive group.

In an eleventh aspect, in addition to one or more of the preceding or following aspects, or in the alternative to some aspects, the photo-reactive polyvinylpyrrolidone compound can include a benzophenone group.

In a twelfth aspect, in addition to one or more of the preceding or following aspects, or in the alternative to some aspects, the non-fouling basecoat layer can be semi-permeable.

In a thirteenth aspect, in addition to one or more of the preceding or following aspects, or in the alternative to some aspects, the non-fouling basecoat layer can be permeable to glucose.

In a fourteenth aspect, in addition to one or more of the preceding or following aspects, or in the alternative to some aspects, the substrate can include a sensor interface.

In a fifteenth aspect, in addition to one or more of the preceding or following aspects, or in the alternative to some aspects, the sensor interface can include an electrochemical sensor interface.

In a sixteenth aspect, in addition to one or more of the preceding or following aspects, or in the alternative to some aspects, the medical device can be an implantable glucose sensor.

In a seventeenth aspect, in addition to one or more of the preceding or following aspects, or in the alternative to some aspects, the lubricious topcoat layer can further include a heparin compound.

In an eighteenth aspect, in addition to one or more of the preceding or following aspects, or in the alternative to some aspects, the heparin compound can include a photoreactive group.

In a nineteenth aspect, in addition to one or more of the preceding or following aspects, or in the alternative to some aspects, the heparin compound can be cross-linked with the photo-reactive polyvinylpyrrolidone compound.

In a twentieth aspect, in addition to one or more of the preceding or following aspects, or in the alternative to some aspects, the heparin compound can include an elutable heparin compound, wherein the elutable heparin compound can be ionically complexed with a polycationic polymer.

In a twenty-first aspect, in addition to one or more of the preceding or following aspects, or in the alternative to some aspects, the polycationic polymer can include at least one selected from the group consisting of PEI, PHEMA-co-DMAEMA, and PEG-DMAEMA.

In a twenty-second aspect, in addition to one or more of the preceding or following aspects, or in the alternative to some aspects, the lubricious topcoat layer can further include a non-photoreactive polyvinylpyrrolidone.

In a twenty-third aspect, in addition to one or more of the preceding or following aspects, or in the alternative to some aspects, the cross-linking agent can include a photoreactive compound.

In a twenty-fourth aspect, in addition to one or more of the preceding or following aspects, or in the alternative to some aspects, the photoreactive compound can include a benzophenone group.

In a twenty-fifth aspect, in addition to one or more of the preceding or following aspects, or in the alternative to some aspects, the cross-linking agent can include a photoreactive phosphate compound.

In a twenty-sixth aspect, in addition to one or more of the preceding or following aspects, or in the alternative to some aspects, the cross-linking agent can include bis(4-benzoylpheny) phosphate of a salt thereof.

In a twenty-seventh aspect, in addition to one or more of the preceding or following aspects, or in the alternative to some aspects, the cross-linking agent can include sodium bis(4-benzoylpheny) phosphate.

In a twenty-eighth aspect, in addition to one or more of the preceding or following aspects, or in the alternative to some aspects, the lubricious topcoat layer can further include a polyacrylamide polymer.

In a twenty-ninth aspect, in addition to one or more of the preceding or following aspects, or in the alternative to some aspects, the polyacrylamide polymer can include a polyacrylamide containing copolymer.

In a thirtieth aspect, in addition to one or more of the preceding or following aspects, or in the alternative to some aspects, the polyacrylamide polymer can include N-Acetylated poly[acrylamide-co-sodium-2-acrylamido-2-methylpropanesulfonate-co-N-(3-(4-benzoylbenzamido)propyl)methacrylamide]-co-methoxy poly(ethylene glycol)1000.

In a thirty-first aspect, in addition to one or more of the preceding or following aspects, or in the alternative to some aspects, the polyacrylamide polymer can include polyacrylamide-co-sodium-2-acrylamido-2-methylpropanesulfonate-co-N-(3-(4-benzoylbenzamido)propyl)methacrylamide.

In a thirty-second aspect, in addition to one or more of the preceding or following aspects, or in the alternative to some aspects, the lubricious topcoat layer can further include a polyzwitterion.

In a thirty-third aspect, in addition to one or more of the preceding or following aspects, or in the alternative to some aspects, the polyzwitterion can include at least one selected from the group consisting of a polysulfobetaine (PSB) and a polyphosphoryl choline (PMPC).

In a thirty-fourth aspect, in addition to one or more of the preceding or following aspects, or in the alternative to some aspects, the lubricious topcoat layer can further include a polyethylene oxide (PEO) polymer.

In a thirty-fifth aspect, in addition to one or more of the preceding or following aspects, or in the alternative to some aspects, the polyethylene oxide (PEO) polymer and the polyzwitterion can be covalently bonded with at least one of the photo-reactive polyvinylpyrrolidone compound and a heparin compound.

In a thirty-sixth aspect, in addition to one or more of the preceding or following aspects, or in the alternative to some aspects, the lubricious topcoat layer can further include an anionic polymer.

In a thirty-seventh aspect, in addition to one or more of the preceding or following aspects, or in the alternative to some aspects, the anionic polymer can include at least one selected from the group consisting of polyacrylic acid (PAA), poly(acrylic acid-co-acrylamide) p(AA-co-AAm), and poly(acrylic acid-co-vinyl pyrrolidone) p(AA-co-VP).

In a thirty-eighth aspect, in addition to one or more of the preceding or following aspects, or in the alternative to some aspects, the lubricious topcoat layer the medical device can further include a first layer and a second layer, wherein the second layer can be disposed on the first layer and can be different than the first layer.

In a thirty-ninth aspect, in addition to one or more of the preceding or following aspects, or in the alternative to some aspects, the second layer exhibits a different degree of cross-linking than the first layer.

In a fortieth aspect, in addition to one or more of the preceding or following aspects, or in the alternative to some aspects, the lubricious topcoat layer can further include an elutable antiplatelet macromer.

In a forty-first aspect, in addition to one or more of the preceding or following aspects, or in the alternative to some aspects, the elutable antiplatelet macromer can include at least one selected from the group consisting of a PEO polymer, a PEO-PPO-PEO copolymer (Pluronics), and a polyvinylpyrrolidone polymer.

In a forty-second aspect, in addition to one or more of the preceding or following aspects, or in the alternative to some aspects, the lubricious topcoat layer can further include a non-prodrug anti-platelet agent.

In a forty-third aspect, in addition to one or more of the preceding or following aspects, or in the alternative to some aspects, can further include a wearable patch.

In a forty-fourth aspect, a thromboresistant coating for an implantable, partially implantable, or wearable medical device can be included having a non-fouling basecoat layer. The non-fouling basecoat layer can include a hydrophilic component and a hydrophobic component. The coating can also include a lubricious topcoat layer. The lubricious topcoat layer can include a photo-reactive polyvinylpyrrolidone and a cross-linking agent, and wherein the lubricious topcoat layer can be disposed over the non-fouling basecoat layer.

In a forty-fifth aspect, in addition to one or more of the preceding or following aspects, or in the alternative to some aspects, the photo-reactive polyvinylpyrrolidone can include a benzophenone group.

In a forty-sixth aspect, in addition to one or more of the preceding or following aspects, or in the alternative to some aspects, the hydrophobic component can include a poly(butyl methacrylate).

In a forty-seventh aspect, in addition to one or more of the preceding or following aspects, or in the alternative to some aspects, the hydrophobic component can include a poly(n-butyl methacrylate).

In a forty-eighth aspect, in addition to one or more of the preceding or following aspects, or in the alternative to some aspects, the hydrophilic component can include a polyvinylpyrrolidone polymer.

In a forty-ninth aspect, in addition to one or more of the preceding or following aspects, or in the alternative to some aspects, the hydrophilic component can include a cross-linked polyvinylpyrrolidone.

In a fiftieth aspect, in addition to one or more of the preceding or following aspects, or in the alternative to some aspects, the hydrophilic component can include an acrylamide polymer, wherein a polyvinylpyrrolidone polymer and the acrylamide polymer can be cross-linked.

In a fifty-first aspect, in addition to one or more of the preceding or following aspects, or in the alternative to some aspects, the non-fouling basecoat layer can further include a heparin compound.

In a fifty-second aspect, in addition to one or more of the preceding or following aspects, or in the alternative to some aspects, the heparin compound can include a photoreactive group.

In a fifty-third aspect, in addition to one or more of the preceding or following aspects, or in the alternative to some aspects, the non-fouling basecoat layer can be semi-permeable.

In a fifty-fourth aspect, in addition to one or more of the preceding or following aspects, or in the alternative to some aspects, the non-fouling basecoat layer can be permeable to glucose.

In a fifty-fifth aspect, in addition to one or more of the preceding or following aspects, or in the alternative to some aspects, can further include an implantable glucose sensor.

In a fifty-sixth aspect, in addition to one or more of the preceding or following aspects, or in the alternative to some aspects, the lubricious topcoat layer can further include a heparin compound.

In a fifty-seventh aspect, in addition to one or more of the preceding or following aspects, or in the alternative to some aspects, the heparin compound can include a photoreactive group.

In a fifty-eighth aspect, in addition to one or more of the preceding or following aspects, or in the alternative to some aspects, the heparin compound can be cross-linked with the photo-reactive polyvinylpyrrolidone.

In a fifty-ninth aspect, in addition to one or more of the preceding or following aspects, or in the alternative to some aspects, the heparin compound can include an elutable heparin compound, wherein the elutable heparin compound can be ionically complexed with a polycationic polymer.

In a sixtieth aspect, in addition to one or more of the preceding or following aspects, or in the alternative to some aspects, the polycationic polymer can include at least one selected from the group consisting of PEI, PHEMA-co-DMAEMA, and PEG-DMAEMA.

In a sixty-first aspect, in addition to one or more of the preceding or following aspects, or in the alternative to some aspects, the lubricious topcoat layer can further include a non-photoreactive polyvinylpyrrolidone.

In a sixty-second aspect, in addition to one or more of the preceding or following aspects, or in the alternative to some aspects, the cross-linking agent can include a photoreactive compound.

In a sixty-third aspect, in addition to one or more of the preceding or following aspects, or in the alternative to some aspects, the photoreactive compound can include a benzophenone group.

In a sixty-fourth aspect, in addition to one or more of the preceding or following aspects, or in the alternative to some aspects, the cross-linking agent can include a photoreactive phosphate compound.

In a sixty-fifth aspect, in addition to one or more of the preceding or following aspects, or in the alternative to some aspects, the cross-linking agent can include bis(4-benzoylpheny) phosphate of a salt thereof.

In a sixty-sixth aspect, in addition to one or more of the preceding or following aspects, or in the alternative to some aspects, the cross-linking agent can include sodium bis(4-benzoylpheny) phosphate.

In a sixty-seventh aspect, in addition to one or more of the preceding or following aspects, or in the alternative to some aspects, the lubricious topcoat layer can further include a polyacrylamide polymer.

In a sixty-eighth aspect, in addition to one or more of the preceding or following aspects, or in the alternative to some aspects, the polyacrylamide polymer can include a polyacrylamide containing copolymer.

In a sixty-ninth aspect, in addition to one or more of the preceding or following aspects, or in the alternative to some aspects, the polyacrylamide polymer can include N-Acetylated poly[acrylamide-co-sodium-2-acrylamido-2-methylpropanesulfonate-co-N-(3-(4-benzoylbenzamido)propyl)methacrylamide]-co-methoxy poly(ethylene glycol)1000.

In a seventieth aspect, in addition to one or more of the preceding or following aspects, or in the alternative to some aspects, the polyacrylamide polymer can include polyacrylamide-co-sodium-2-acrylamido-2-methylpropanesulfonate-co-N-(3-(4-benzoylbenzamido)propyl)methacrylamide.

In a seventy-first aspect, in addition to one or more of the preceding or following aspects, or in the alternative to some aspects, the lubricious topcoat layer can further include a polyzwitterion.

In a seventy-second aspect, in addition to one or more of the preceding or following aspects, or in the alternative to some aspects, the polyzwitterion can include at least one selected from the group consisting of a polysulfobetaine (PSB) and a polyphosphoryl choline (PMPC).

In a seventy-third aspect, in addition to one or more of the preceding or following aspects, or in the alternative to some aspects, the lubricious topcoat layer can further include a polyethylene oxide (PEO) polymer.

In a seventy-fourth aspect, in addition to one or more of the preceding or following aspects, or in the alternative to some aspects, the polyethylene oxide (PEO) polymer and the polyzwitterion can be covalently bonded with at least one of the photo-reactive polyvinylpyrrolidone and a heparin compound.

In a seventy-fifth aspect, in addition to one or more of the preceding or following aspects, or in the alternative to some aspects, the lubricious topcoat layer can further include an anionic polymer.

In a seventy-sixth aspect, in addition to one or more of the preceding or following aspects, or in the alternative to some aspects, the anionic polymer can include at least one selected from the group consisting of polyacrylic acid (PAA), poly(acrylic acid-co-acrylmide) p(AA-co-AAm), and poly(acrylic acid-co-vinyl pyrrolidone) p(AA-co-VP).

In a seventy-seventh aspect, in addition to one or more of the preceding or following aspects, or in the alternative to some aspects, the lubricious topcoat layer the thromboresistant coating can further include a first layer, and a second layer, wherein the second layer can be disposed on the first layer and can be different than the first layer.

In a seventy-eighth aspect, in addition to one or more of the preceding or following aspects, or in the alternative to some aspects, the second layer exhibits a different degree of cross-linking than the first layer.

In a seventy-ninth aspect, in addition to one or more of the preceding or following aspects, or in the alternative to some aspects, the lubricious topcoat layer can further include an elutable antiplatelet macromer.

In an eightieth aspect, in addition to one or more of the preceding or following aspects, or in the alternative to some aspects, the elutable antiplatelet macromer can include at least one selected from the group consisting of a PEO polymer, a PEO-PPO-PEO copolymer (Pluronics), and a polyvinylpyrrolidone polymer.

In an eighty-first aspect, in addition to one or more of the preceding or following aspects, or in the alternative to some aspects, the lubricious topcoat layer can further include a non-prodrug anti-platelet agent.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a coated medical device in accordance with various embodiments herein.

FIG. 2 is a schematic view of a coated medical device in accordance with various embodiments herein.

FIG. 3 is a schematic view of a coated medical device in accordance with various embodiments herein.

FIG. 4 is a cross-sectional view of a portion of a coated medical device in accordance with various embodiments herein.

FIG. 5 is a cross-sectional view of a portion of a coated medical device in accordance with various embodiments herein.

FIG. 6 is a cross-sectional view of a portion of a coated medical device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Certain medical devices such as implantable, partially implantable, or wearable devices in contact with portions of the body can be susceptible to biofouling. Biofouling can interfere with the performance of such medical devices including therapeutic functions thereof, sensing functions thereof, or the like and lead to other adverse outcomes.

Embodiments herein include thromboresistant coatings that can be disposed on medical devices (such as on a substrate thereof, a sensor thereof, an array of sensors, a sensor interface thereof, an electrode thereof, or the like) and can resist biofouling thereby enhancing the performance of the medical device over time. In particular, sensor systems coated herein can include multi-sensor systems and/or sensor arrays.

In use, an analyte (such as a metabolite or other compound) diffuses across a coating to the sensor. These sensor systems use measurements from the different sensors or sensor heads to provide a desired/accurate measurement (e.g., blood glucose). Coatings can be used to protect the sensors. However, conventional coatings can interfere with the dilution of the metabolite to the sensor and/or cause the metabolite to diffuse to each sensor at a different rate, which impacts the accuracy of the measurement.

Coatings herein can combine a topcoat with a basecoat having a hydrophilic component and a hydrophobic component. The topcoat provides a lubricious surface and protects the coating. The basecoat ties the topcoat to a substrate. In addition, the mix of hydrophilic and hydrophobic polymers in the basecoat can act as a rate limiter controlling the diffusion rate of the metabolite across the coating. Specifically, the hydrophilic component helps the metabolite diffuse across the basecoat while the hydrophobic polymer acts as a rate limiter slowing the diffusion. While not intending to be bound by theory, it has been found herein that a specific ratio of hydrophilic and hydrophobic polymers helps to provide a more consistent rate of transfer between all the sensors. In contrast, when the ratio is off the metabolite diffuses to the sensors at different rates creating accuracy issues. Heparin or other anti-fouling coating components can also be included in the basecoat to help maintain consistent diffusion of the metabolite.

In various embodiments, the topcoat can include a photo-reactive polyvinylpyrrolidone compound and a cross-linking agent, such as bis(4-benzoylpheny) phosphate. In various embodiments, the basecoat can include a polyvinylpyrrolidone compounds as a hydrophilic component and polybutylmethacrylate compound as a hydrophobic component.

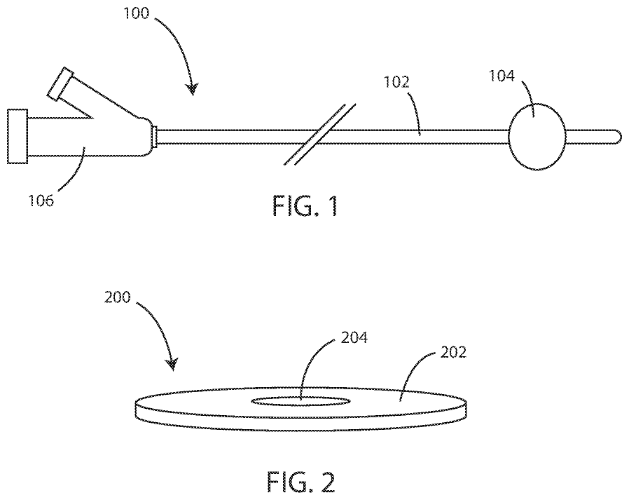

Referring now to FIG. 1, a schematic view of a coated medical device 100 is shown in accordance with various embodiments herein. The medical device 100 includes a shaft 102, a balloon 104, and a proximal manifold 106. Coatings herein can be disposed on any portion of the medical device 100 including, but not limited to, the shaft 102, the balloon 104, the proximal manifold 106, or other portions thereof.

In various embodiments, coatings herein can specifically be disposed over a sensor or a portion thereof. Referring now to FIG. 2, a schematic view of a coated medical device 200 is shown in accordance with various embodiments herein. The medical device can be a wearable patch. The wearable patch can include a housing 202 and a sensor 204. The sensor 204 can be of various types including, for example, an optical sensor, an electrochemical sensor, an electrical potential sensor, or the like. Coatings herein can be disposed over the sensor 204, amongst other portions of the medical device 200. For example, coatings herein can be disposed over a sensor interface (or portion of the senor interfacing with tissues and/or fluids of the body) of the sensor 204.

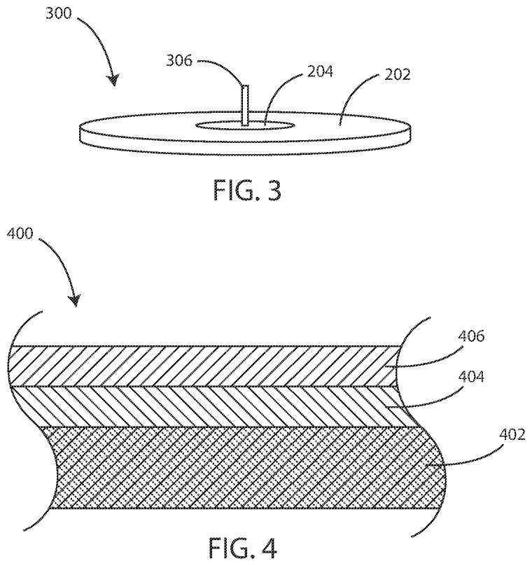

Referring now to FIG. 3, a schematic view of a coated medical device 300 is shown in accordance with various embodiments herein. The coated medical device 300 can include a housing 202, a sensor 204, and a needle 306. In this example, the needle 306 can be used to provide contact with a tissue or fluid of the body for measurement such as contact with blood, lymph fluid, interstitial fluid, or the like. The use of a needle is merely one example of a structural feature which can be used to provide contact with a tissue or body. Coatings herein can be disposed over the sensor 204, the needle 306 (or another contact structure), or other portions of the medical device 300.

Referring now to FIG. 4, a cross-sectional view of a portion of a coated medical device 400 is shown in accordance with various embodiments herein. The coated device includes a substrate 402, which can be a polymer, a metal, a composite, a ceramic, or the like. The medical device also includes a barrier membrane 404. The barrier membrane 404 can be disposed over the substrate 402. Further details of exemplary barrier membranes are provided in greater detail below. The medical device also includes a non-fouling, tissue compatible coating 406 or portion disposed over the membrane 404. Further details of exemplary non-fouling, tissue compatible coatings are provided below.

In various embodiments, the barrier membrane 404 can be disposed over a substrate 402. In various embodiments, the barrier membrane 404 can be semi-permeable. In various embodiments, the barrier membrane 404 can be permeable to glucose or other biological solutes. In various embodiments, the non-fouling, tissue compatible coating 406 can be disposed over the barrier membrane 404.

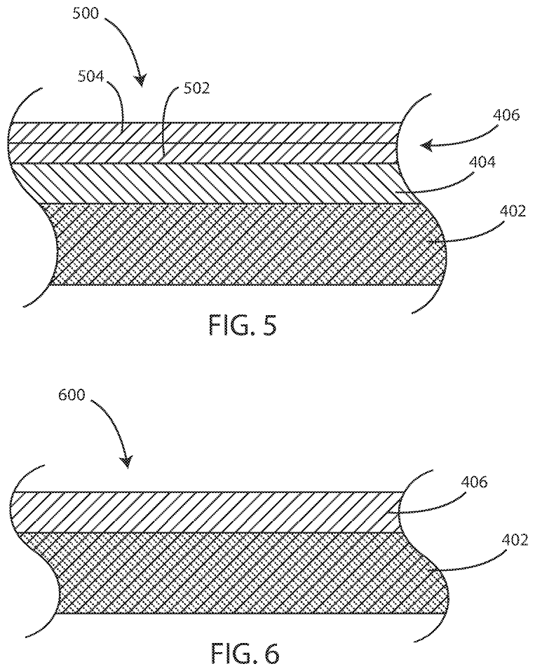

In some embodiments the non-fouling, tissue compatible coating can be in the form of multiple layers. Referring now to FIG. 5, a cross-sectional view of a portion of a coated medical device 500 is shown in accordance with various embodiments herein. The coated device 500 includes a substrate 402, a barrier membrane 404 disposed over the substrate 402, and a non-fouling, tissue compatible coating 406 disposed over the barrier membrane 404. The non-fouling, tissue compatible coating 406 includes a first layer 502 and a second layer 504. In various embodiments, the second layer 504 can be different than the first layer 502. In various embodiments, for example, the second layer 504 exhibits a different degree of cross-linking than a first layer 502.

In various embodiments, one or more portions or segments of the coating can be omitted. For example, in some embodiments the barrier membrane 404 can be omitted. Referring now to FIG. 6, a cross-sectional view of a portion of a coated medical device 600 is shown in accordance with various embodiments herein. The coated medical device 600 includes a substrate 402 and a non-fouling, tissue compatible coating 406 disposed over the substrate 402.

### Barrier Membrane/Basecoat

Various embodiments herein include a barrier membrane or basecoat. Further details about the barrier membrane are provided as follows. However, it will be appreciated that this is merely provided by way of example and that further variations are contemplated herein.

In various embodiments, the barrier membrane is semi-permeable. In various embodiments, the barrier membrane is permeable to various biological solutes and/or therapeutic agents. In various embodiments, the barrier membrane is impermeable to larger molecules such as proteins. In various embodiments, the barrier membrane is specifically permeable to glucose. In various embodiments, the barrier membrane is specifically permeable to various physiological electrolytes. However, in other embodiments the barrier membrane (where included) is substantially impermeable.

The basecoat can serve various purposes. For example, the basecoat ties the topcoat to a substrate. In addition, the basecoat can include a mix of hydrophilic and hydrophobic polymers and can act as a rate limiter controlling the diffusion rate of the metabolite across the coating. Specifically, the hydrophilic component helps the metabolite diffuse across the basecoat while the hydrophobic polymer acts as a rate limiter slowing the diffusion.

While not intending to be bound by theory, it has been found herein that a specific ratio of hydrophilic and hydrophobic polymers helps to provide a more consistent rate of transfer between all the sensors. In contrast, when the ratio is off the metabolite diffuses to the sensors at different rates creating accuracy issues. In various embodiments, more of the hydrophobic polymer is used than the hydrophilic polymer. In various embodiments, the ratio (by weight) of the hydrophobic polymer (such as poly(n-butyl methacrylate) or another hydrophobic polymer such as those described herein) to the hydrophilic polymer (such as a polyvinylpyrrolidone polymer or copolymer or another hydrophilic polymer such as those described herein) can be about from about 1:1 to 8:1, or from about 2:1 to about 6:1, or from about 2:1 to about 5:1, or from about 2:1 to about 4:1, or from about 2.5:1 to about 3.5:1, or about 3:1.

In various embodiments, the barrier membrane can include permeable polymer layer. In various embodiments, the barrier membrane can include various acrylates or methacrylates. In various embodiments, the barrier membrane can include a poly(butyl methacrylate). In various embodiments, the barrier membrane can specifically include a poly(n-butyl methacrylate).

In various embodiments, the barrier membrane can include a polyvinylpyrrolidone polymer and/or a polyacrylamide polymer. In various embodiments, the barrier membrane can include a cross-linked polyvinylpyrrolidone polymer. In various embodiments, the barrier membrane can include a cross-linked polyacrylamide polymer. In various embodiments, the barrier membrane can include a polyvinylpyrrolidone polymer and an acrylamide polymer, wherein the polyvinylpyrrolidone polymer and the acrylamide polymer are covalently bonded to one another. In various embodiments, the barrier membrane can include a polyvinylpyrrolidone polymer and an acrylamide polymer, wherein the polyvinylpyrrolidone polymer and the acrylamide polymer are cross-linked forming a cross-linked polymeric matrix. In some embodiments, the barrier membrane can include the reaction product of a cross-linking agent serving to provide cross-links. Exemplary cross-linking agents are described in greater detail below.

In various embodiments, the barrier membrane can include a heparin compound or other similar agents. In some embodiments the heparin compound can be covalently bonded to other components of the barrier membrane. In some embodiments the heparin compound can be a photo-reactive heparin compound (e.g., a heparin compound modified to include a photoreactive group, such as a benzophenone group). In some embodiments the heparin compound can be non-covalently bonded to other components of the barrier membrane. In some embodiments the heparin compound can be configured to elute out from the barrier membrane. Further details of exemplary heparin compounds are provided in greater detail below.

While not intending to be bound by theory, it is believed that applying a specialized layer or base coat over a substrate (in addition to or in place of a barrier membrane as described herein) can aid in modulating a thromboresistant coating or layer herein for enhanced biological function of thromboresistance. By way of example, a specialized layer or base coat herein can be applied over nickel-titanium, stainless steel, cobalt-chromium, or other device surfaces. In some embodiments, a specialized layer or base coat can include materials described herein with respect to the barrier membrane such as a poly(butyl methacrylate). In some embodiments, a specialized layer or base coat can include a fluoropolymer (homopolymer or copolymer). Exemplary fluoropolymers or copolymers and include solvent-processed, plasma-deposited, physical vapor-deposited polymers or the like (and can also include those applied using other techniques). Exemplary fluoropolymers can include, but are not limited to, PVDF, PTFE, PVDF-co-HFP, terpolymer THV (PVDF-co-HFP-Co-TFE) and the like.

In some embodiments, the coatings herein, including but not limited to the barrier membrane or base coat, can be a drug eluting coating. For example, the barrier membrane or base coat or other components of the coating can include various active agents such as mTOR inhibitors, everolimus, sirolimus (rapamycin), tacrolimus, zotarolimus, rapalogs, paclitaxel, or the like.

Various solvents can be used to apply the basecoat. However, in some embodiments, the solvent can include THF and/or with an alcohol such as isopropyl alcohol.

### Non-Fouling, Tissue Compatible Coating/Topcoat

Various embodiments herein include a non-fouling, tissue compatible coating. Further details about the non-fouling, tissue compatible coating are provided as follows. However, it will be appreciated that this is merely provided by way of example and that further variations are contemplated herein.

The non-fouling, tissue compatible coating can be composed of various materials. In various embodiments, the non-fouling, tissue compatible coating can include a polyvinylpyrrolidone polymer. In various embodiments, the topcoat can include topcoat can include a polyvinylpyrrolidone compound (such as a photo-reactive PVP compound) and a cross-linking agent, such as bis(4-benzoylpheny) phosphate.

In various embodiments, the non-fouling, tissue compatible coating can also include a heparin compound.

In various embodiments, the non-fouling, tissue compatible coating can include a cross-linking agent, and a polyacrylamide polymer.

In various embodiments, the non-fouling, tissue compatible coating can include a polyzwitterion. In various embodiments, the polyzwitterion can include at least one including at least one of a polysulfobetaine (PSB), polysulfobetaine methacrylate (PSBMA), and a polyphosphoryl choline (PMPC). In some embodiments, polyzwitterions herein can be photoderivatized with a photoreactive group, but in other embodiments are non-photoderivatized.

In various embodiments, the non-fouling, tissue compatible coating can include a polyethylene oxide (PEO) polymer. In some embodiments, PEO polymers herein can be photoderivatized with a photoreactive group, but in other embodiments are non-photoderivatized.

In various embodiments, the polyethylene oxide (PEO) polymer and the polyzwitterion are covalently bonded with at least one of the polyvinylpyrrolidone and the heparin compound.

In various embodiments, the non-fouling, tissue compatible coating can include an anionic polymer. In various embodiments, the anionic polymer can include at least one including at least one of polyacrylic acid (PAA), poly(acrylic acid-co-acrylamide) (AA-co-AAm), and poly(acrylic acid-co-vinyl pyrrolidone) (AA-co-VP).

In various embodiments, the non-fouling, tissue compatible coating can include an elutable antiplatelet macromer. In various embodiments, the non-fouling, tissue compatible coating can include a non-prodrug anti-platelet agent.

Various specific embodiments of the non-fouling, tissue compatible coating include PVP in combination with: 1.) PSBMA only, 2.) PEO only, 3.) PSBMA+Heparin, 4.) PSBMA+PEO +Heparin, 5.) PEO+Heparin, and 6.) PSBMA+PEO.

Various solvents can be used to apply the topcoat. However, in some embodiments, the solvent can include an alcohol (such as methanol) and/or water.

### Heparin Compounds

Various embodiments herein include an elutable heparin compound. Further details about the elutable heparin compound are provided as follows. However, it will be appreciated that this is merely provided by way of example and that further variations are contemplated herein.

Heparin compounds herein can include heparin, heparin derivatives, sodium heparin, low molecular weight heparin, high affinity heparin, low affinity heparin, and the like.

In various embodiments, the heparin compound can include a photoreactive group. For instance, photoderivatized heparin (“photoheparin”) can be prepared by those skilled in the art, in the manner described in U.S. Pat. No. 5,563,056 (Swan et al., Preparation of Crosslinked Matrices Containing Covalently Immobilized Chemical Species and Unbound Releasable Chemical Species), which describes preparation of photoheparin by reacting heparin with benzoyl-benzoyl-epsilon-aminocaproyl-N-oxysuccinimide in dimethylsulfoxide/carbonate buffer. The solvent was evaporated and the photoheparin was dialyzed against water, lyophilized, and then dissolved in water.

In various embodiments, the heparin compound is cross-linked with the polyvinylpyrrolidone. Heparin can be cross-linked with other compounds through various techniques including, for example, using a cross-linking agent as described herein.

In various embodiments, the heparin compound can include an elutable heparin compound, wherein the elutable heparin compound is ionically complexed with a polycationic polymer. Elutable heparin compounds are not covalently bonded into fixed portions of the coating so that they can elute out.

### Polycationic Polymers

Various embodiments herein include a polycationic polymer. Further details about exemplary polycationic polymers are provided as follows. However, it will be appreciated that this is merely provided by way of example and that further variations are contemplated herein.

In general, polycationic polymers include any polymer containing cationic groups and/or groups which may be ionized into cationic groups. The polycationic polymer can be selected from those that contain units comprising primary, secondary, tertiary and/or quaternary amine groups which may either form part of the main polymer chain or may be side substituents linked to the main chain. In various embodiments, the polycationic polymer contains cationic nitrogen-containing moieties such as quaternary ammonium or cationic protonated amino moieties. The cationic protonated amines can be primary, secondary, or tertiary amines, depending upon the particular species and the selected pH of the composition.

In various embodiments, the polycationic polymer can specifically include at least one including at least one of polyethyleneimine (PEI) homopolymers and copolymers, copolymers of poly(2-hydroxyethyl methacrylate) and dimethylaminoethyl methacrylate (DMAEMA) (e.g., pHEMA-co-DMAEMA), and copolymers of polyethylene glycol and dimethylaminoethyl methacrylate (PEG-DMAEMA).

While not intending to be bound by theory, it is believed that such polycationic polymers can offer benefits, particularly in the contents of embodiments herein including heparin. In specific, heparin can be ionically complexed with a polycationic homopolymer or a polycationic copolymer. For example, a coating herein can include PEI or PHEMA-co-DMAEMA or PEG- DMAEMA that will complex with the non-photo heparin. Upon presenting to the blood, or other fluid or tissue, de-complexation can result in slow leaching of heparin that can reduce the potency of the geometry-activated platelets.

### Anionic Polymers

In various embodiments, the non-fouling, tissue compatible coating can include an anionic polymer. In various embodiments, the anionic polymer can include polyacrylic acid subunits. In various embodiments, the anionic polymer can include at least one including at least one of polyacrylic acid (PAA) homopolymer or copolymers, poly(acrylic acid-co-acrylamide) (AA-co-AAm), and poly(acrylic acid-co-vinyl pyrrolidone) (AA-co-VP).

While not intending to be bound by theory, it is believed that anionic polymers (including, for example, PAA homopolymer or PAA subunit containing copolymers) adds to thromboresistance. In addition, anionic polymers herein (including, for example, PAA homopolymer or PAA subunit containing copolymers) can allow/facilitate extractable or elutable components herein including one or more of polyethylene oxide polymers (PEO), polysulfobetaine methacrylate polymers (PSBMA), heparin compounds or the like so that they release slowly into the blood or other fluid.

### Polyvinylpyrrolidone Polymers

Various embodiments herein include a polyvinylpyrrolidone polymer. Further details about the polyvinylpyrrolidone are provided as follows. However, it will be appreciated that this is merely provided by way of example and that further variations are contemplated herein.

Polyvinylpyrrolidone polymers herein can include polyvinylpyrrolidone homopolymers as well as polyvinylpyrrolidone subunit containing copolymers. By way of example, polyvinylpyrrolidone copolymers can include subunits of polyvinylpyrrolidone as follows:

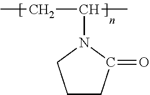

Polyvinylpyrrolidone polymers herein can be linear or can be branched. Polyvinylpyrrolidone polymers herein can have various molecular weights such as an average molecular weight from 1 kDa to 3000 kDa. In various embodiments, the polyvinylpyrrolidone polymer has an average molecular weight from 10 kDa to 50 kDa. In various embodiments, the lubricious coated medical device 100 wherein a non-photoreactive polyvinylpyrrolidone is a blend of different molecular weight PVP compounds. Exemplary non-photo derivatized polyvinylpyrrolidone polymers can include, for example, PVP K12, PVP K30, PVP K90, and the like.

Polyvinylpyrrolidone polymers herein can be used to form a PVP hydrogel.

In various embodiments, the polyvinylpyrrolidone can include a non-photoreactive polyvinylpyrrolidone. However, instead of or in addition to non-photoreactive polyvinylpyrrolidone, in various embodiments, the polyvinylpyrrolidone can also include a photoreactive polyvinylpyrrolidone. Photoreactive polyvinylpyrrolidones can include homopolymers and/or copolymers where they are derivatized to include a photoreactive group. In various embodiments, the photoreactive polyvinylpyrrolidone can specifically include a benzophenone group.

An exemplary photoreactive polyvinylpyrrolidone copolymer can include poly[vinyl pyrrolidone-co-N-(3-(4-benzoylbenzamideo)propyl)methacrylamide] (or PVP-co-APMA with 80 to 99.9 mole percent PVP and 20 to 0.1 mole percent APMA). By way of example, an exemplary photoreactive polyvinylpyrrolidone is as follows:

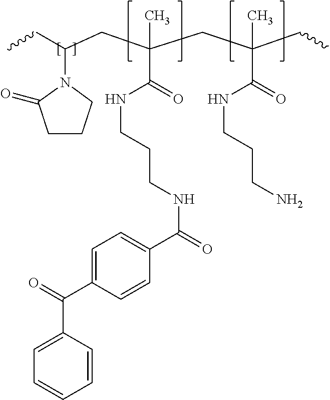

Another exemplary photoreactive polyvinylpyrrolidone copolymer (acetylated PVP-APMA-BBA; or acetylated photo-PVP) is as follows:

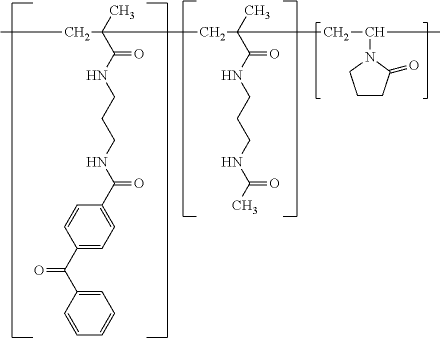

This compound can be prepared by a copolymerization of 1-vinyl-2-pyrrolidone and N-(3-aminopropyl)methacrylamide (APMA,follwed by photoderivatization of the polymer using 4-benzoylbenzoyl chloride under Schotten-Baumann conditions. The unreacted amines of the photopolymer can be further acetylated using acetic anhydride.

### Polyacrylamide Polymers

Various embodiments herein include a polyacrylamide polymer. Further details about the polyacrylamide polymer are provided as follows. However, it will be appreciated that this is merely provided by way of example and that further variations are contemplated herein.

In various embodiments, the polyacrylamide polymer can include a polyacrylamide homopolymer. In various embodiments, the polyacrylamide polymer can include a polyacrylamide containing copolymer.

In various embodiments, the polyacrylamide polymer can include a photo-derivatized polyacrylamide polymer (e.g., a polyacrylamide polymer or copolymer modified to include a photoreactive group). In various embodiments, the polyacrylamide polymer can include a non-photo-derivatized polyacrylamide polymer.

In some embodiments, the polyacrylamide polymers herein can also include acrylamido-2-methylpropanesulfonate groups (AMPS) and polyethyleneglycol segments. In a specific embodiment, the polymer comprising polyacrylamide can be N-Acetylated poly[acrylamide-co-sodium-2-acrylamido-2-methylpropanesulfonate-co-N-(3-(4-benzoylbenzamido)propyl)methacrylamide]-co-methoxy poly(ethylene glycol) monomethacrylate. Polymers comprising polyacrylamide in accordance with embodiments herein are described in U.S. Pat. Nos. 4,979,959; 5,263,992; and 5,512,329, the content of all of which is herein incorporated by reference in its entirety.

In various embodiments, the polyacrylamide polymer can specifically include N-Acetylated poly[acrylamide-co-sodium-2-acrylamido-2-methylpropanesulfonate-co-N-(3-(4-benzoylbenzamido)propyl)methacrylamide]-co-methoxy poly(ethylene glycol)1000.

In various embodiments, the polyacrylamide polymer can specifically include polyacrylamide-co-sodium-2-acrylamido-2-methylpropanesulfonate-co-N-(3-(4-benzoylbenzamido)propyl)methacrylamide.

### Elutable Antiplatelet Agents

Various embodiments herein include an elutable antiplatelet agent. For example, various embodiments herein can include an elutable antiplatelet macromer. Further details about the elutable antiplatelet agents and/or macromers are provided as follows.

However, it will be appreciated that this is merely provided by way of example and that further variations are contemplated herein.

In various embodiments, the elutable antiplatelet macromer can include at least one including at least one of a polyethylene oxide (PEO) polymer, a Pluronics polymer (poloxamers/PEO-PPO-PEO copolymers), and/or a polyvinylpyrrolidone polymer. Such macromers, in order to facilitate being elutable, are generally not cross-linked or otherwise covalently bonded with other components of the coating, layer, or matrix in which they are disposed. While not intending to be bound by theory, it is believed that such elutable macromers can reduce the activity of geometry-activated platelets.

In various embodiments, elutable antiplatelet agents can include a non-prodrug anti-platelet agents. Elutable antiplatelet agents can include, but are not limited to, cyclooxygenase inhibitors, adenosine diphosphate (ADP) receptor inhibitors, phosphodiesterase inhibitors, protease-activated receptor-1 (PAR-1) antagonists, P2Y₁₂ inhibitors (such as CANGRELOR), glycoprotein IIB/IIIA inhibitors, adenosine reuptake inhibitors, thromboxane inhibitors, and the like. Such elutable antiplatelet agents can be used in combination with elutable antiplatelet macromer in some embodiments or can be used independently in other embodiments.

### Cross-Linking Agents

Various embodiments herein include a cross-linking agent. Further details about the cross-linking agent are provided as follows. However, it will be appreciated that this is merely provided by way of example and that further variations are contemplated herein. Further exemplary cross-linking agents are described in U.S. Publ. Pat. App. No. 2011/0245367, the content of which is herein incorporated by reference in its entirety.

In some embodiments, the crosslinking agent(s) can have a molecular weight of less than about 1500 kDa, but in other embodiments can be larger. In some embodiments the crosslinking agent can have a molecular weight of less than about 1200, 1100, 1000, 900, 800, 700, 600, 500, or 400 or less, or a molecular weight falling within a range between any of the foregoing.

In various embodiments, cross-linking agents include one or more photoreactive groups attached to a linking group. The cross-linking agent (or linking agent) can be represented by the formula Photo¹-LG-Photo², wherein Photo¹ and Photo² independently represent at least one photoreactive group and LG represents a linking group. The term “linking group” as used herein, refers to a segment or group of molecules configured to connect two or more molecule to each another. In some embodiments, the linking group can include a heteroatom. In some embodiments, the linking group lacks a heteroatom.

In one embodiment, the linking group includes at least one silicon atom. In another embodiment, the linking group includes at least one phosphorus atom.

In some embodiments, the linking group can be a degradable linking group, which in other embodiments the linking group can be a non-degradable linking group. The term “degradable linking group” as used herein, refers to a moiety configured to connect one molecule to another, wherein the linking group is capable of cleavage under one or more conditions. The term “biodegradable” as used herein, refers to degradation in a biological system, and includes for example, enzymatic degradation or hydrolysis. It should be noted that the term “degradable” as used herein includes both enzymatic and non-enzymatic (or chemical) degradation. It is also understood that hydrolysis can occur in the presence of or without an acid or base. In one embodiment, the linking agent is water soluble. In another embodiment, the linking agent is not water soluble.

In various embodiments the linking group can function as a spacer, for example, to increase the distance between the photoreactive groups of the linking agent. For example, in some instances it may be desirable to provide a spacer to reduce steric hindrance that may result between the photoreactive groups, which could interfere with the ability of the photoreactive groups to form covalent bonds with a support surface, or from serving as a photoinitiator for polymerization. As described herein, it is possible to vary the distance between the photoreactive groups, for example, by increasing or decreasing the spacing between one or more photoreactive groups.

As described herein, one or more photoreactive groups can be bound to a linking group by a degradable or a non-degradable linkage. In various embodiments, the degradable linkage between the photoreactive group and the linking group includes at least one heteroatom, including, but not limited to oxygen, nitrogen, selenium, sulfur or a combination thereof. In one embodiment, a photoreactive group, linking group and heteroatom form an ether (R¹—O—R²), wherein R¹ is a photoreactive group and R² is a linking group. In another embodiment, a photoreactive group, linking group and heteroatom form an amine,

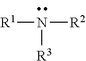

wherein R¹ is a photoreactive group, R² is a linking group, and R³ is hydrogen, aryl or alkyl, a photoreactive group, or a hydroxyl or salt thereof. In one embodiment, R³ is cyclic, linear or branched, saturated or unsaturated, aromatic or heteroaromatic, or a combination thereof. The stability of the ether and/or amine linkage can be influenced depending upon the size (e.g., chain length, branching, bulk, etc.) of the substituents. For example, bulkier substituents will generally result in a more stable linkage (i.e., a linking agent that is slower to degrade in the presence of water and/or acid).

In various embodiments, the linking group includes one or more silicon atoms. In a particular embodiment, the linking group includes one silicon atom (which can be referred to as a monosilane) covalently bound to at least two photoreactive groups. In another embodiment, the linking group includes at least two silicon atoms (which can be referred to as a disilane). In one embodiment, the linking group can be represented by the formula Si—Y—Si, wherein Y represents a linker that can be null (e.g., the linking group includes a direct Si—Si bond), an amine, ether, linear or branched C₁-C₁₀ alkyl, or a combination thereof In one embodiment, Y is selected from O, CH₂, OCH₂CH₂O and O(CH₂CH₂O)ₙ, wherein n is an integer between 1 and 5, between 1 and 10, between 1 and 15, between 1 and 20, between 1 and 25, or between 1 and 30. One embodiment of a disilane linking agent is shown below

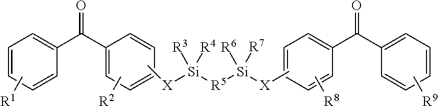

wherein R¹, R², R⁸ and R⁹ can be any substitution, including, but not limited to H, alkyl, halide, hydroxyl, amine, or a combination thereof; R³, R⁴, R⁶ and R⁷ can be alkyl, aryl or a combination thereof R⁵ can be any substitution, including but not limited to O, alkyl or a combination thereof and each X, independently, can be O, N, Se, S, or alkyl, or a combination thereof. One specific embodiment is shown below:

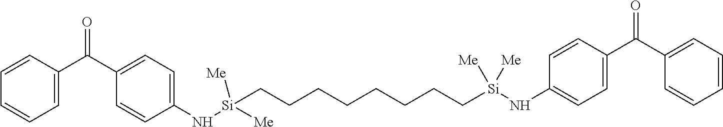 [SMILES: C[Si](C)(CCCCCCCC[Si](C)(C)Nc1ccc(C(=O)c2ccccc2)cc1)Nc1ccc(C(=O)c2ccccc2)cc1]

In various embodiments, the linking agent can be represented by the formula

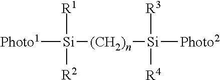

wherein Photo¹ and Photo², independently, represent one or more photoreactive groups and n is an integer between 1 and 10, wherein the linking agent comprises a covalent linkage between at least one photoreactive group and the linking group, wherein the covalent linkage between at least one photoreactive group and the linking group is interrupted by at least one heteroatom. In general, a longer hydrocarbon chain between the two silicon atoms will tend to increase the flexibility of the linking agent and may facilitate crosslinking between a greater number of polymers than a linking agent with a shorter carbon chain, since the photoreactive groups can react with polymers located farther apart from one another. In the formula shown above, R¹, R², R³, R⁴ are independently alkyl or aryl, including, but not limited to cyclic, linear or branched, saturated or unsaturated, aromatic or heteroaromatic, or a combination thereof. In a more particular embodiment, R¹-R⁴ are independently phenyl, methyl, ethyl, isopropyl, t-butyl, or a combination thereof. In another embodiment, R¹-R⁴ can also be, independently, a photoreactive group. In yet another embodiment, R¹-R⁴ can also be, independently, hydroxyl or salt thereof. In one embodiment, the hydroxyl salt includes a counterion that is lithium, sodium, potassium, or a combination thereof.

In another embodiment, the linking agent can be represented by the formula

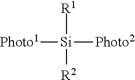

wherein Photo¹ and Photo², independently, represent one or more photoreactive group, wherein the linking agent comprises a covalent linkage between at least one photoreactive group and the linking group, wherein the covalent linkage between at least one photoreactive group and the linking group is interrupted by at least one heteroatom; R¹ and R² are independently alkyl or aryl, including, but not limited to cyclic, linear or branched, saturated or unsaturated, aromatic or heteroaromatic, or a combination thereof. In a more particular embodiment, R¹ and R² are independently phenyl, methyl, ethyl, isopropyl, t-butyl, or a combination thereof. R¹ and R² can also be, independently, a photoreactive group, wherein the linking agent comprises a covalent linkage between at least one photoreactive group and the linking group, wherein the covalent linkage between at least one photoreactive group and the linking group is interrupted by at least one heteroatom; or hydroxyl or salt thereof. In one embodiment, the hydroxyl salt includes a counterion that is lithium, sodium, potassium, or a combination thereof. One embodiment of a monosilane linking agent is shown below

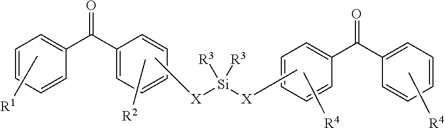

in which R¹ and R⁵ can be any substitution, including, but not limited to H, halogen, amine, hydroxyl, alkyl, or a combination thereof; R² and R⁴ can be any substitution, except OH, including, but not limited to H, alkyl or a combination thereof; R³ can be alkyl, aryl or a combination thereof, including, for example, methyl, ethyl, propyl, isopropyl and butyl; and X, independently, can be O, N, Se, S, alkyl or a combination thereof.

In another embodiment, the linking group includes one or more phosphorous atoms. In one embodiment, the linking group includes one phosphorus atom (which can also be referred to as a mono-phosphorus linking group). In another embodiment, the linking agent includes two phosphorus atoms (which can also be referred to as a bis-phosphorus linking group). In one embodiment, the linking group comprises at least one phosphorus atom with a phosphorus-oxygen double bond (P═O), wherein at least one or two photoreactive groups are bound to the phosphorus atom. In another embodiment, the linking group comprises one phosphorus atom with a phosphorus-oxygen double bond (P═O), wherein two or three photoreactive groups are covalently bound to the phosphorus atom. In another embodiment, the linking group comprises at least two phosphorus atoms, wherein at least one phosphorus atom includes a phosphorus-oxygen double bond (P═O), and at least one or two photoreactive groups are covalently bound to each phosphorus atom.

In a more particular embodiment, the linking agent can be represented by the formula:

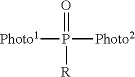

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group, hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In a more particular embodiment, R is phenyl, methyl, ethyl, isopropyl, t-butyl, or a combination thereof.

In another embodiment, the linking agent can be represented by formula:

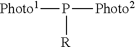

wherein Photo¹ and Photo² independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group (wherein the covalent linkage between the photoreactive group and the linking group may be interrupted by at least one heteroatom), hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In one embodiment, R is phenyl, methyl, ethyl, isopropyl, t-butyl, or a combination thereof.

In another embodiment, the linking agent can be represented by the formula:

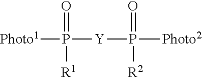

wherein Photo¹ and Photon², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom; Y represents a linker that can be null (i.e., not present, such that the linking group includes a direct P-P bond), N or O, linear or branched C₁-C₁₀ alkyl, or a combination thereof; and R¹ and R² are independently alkyl, aryl, a photoreactive group (wherein the covalent linkage between the photoreactive group and the linking group can be interrupted by at least one heteroatom), hydroxyl or salt thereof, or a combination thereof. In one embodiment, Y is selected from O, CH₂, OCH₂O, OCH₂CH₂O and O(CH₂CH₂O)ₙ, wherein n is an integer between 1 and 5, between 1 and 10, between 1 and 15, between 1 and 20, between 1 and 25, or between 1 and 30. In one embodiment, the hydroxyl salt counterion is lithium, sodium, potassium, or a combination thereof. In a more particular embodiment, R¹ and R² are independently, cyclic, linear or branched hydrocarbon, saturated or unsaturated, aromatic or heteroaromatic, or a combination thereof. In one embodiment, R¹ and R² are independently phenyl, methyl, ethyl, isopropyl, t-butyl, or a combination thereof. In general, a longer hydrocarbon chain between the two phosphorus atoms will tend to increase the flexibility of the linking agent and may facilitate crosslinking between a greater number of polymers than a linking agent with a shorter carbon chain, since the reactive photoreactive groups can react with polymers located farther apart from one another. In one embodiment, Y can be O, CH₂, OCH₂CH₂O and O(CH₂CH₂O)ₙ wherein n is an integer between 1 and 5, between 1 and 10, between 1 and 15, between 1 and 20, between 1 and 25, or between 1 and 30. One embodiment is shown below

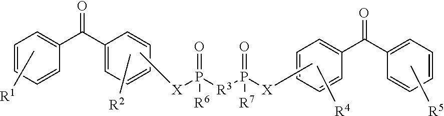

in which R¹, R², R⁴ and R⁵ can be any substitution, including but not limited to H, alkyl, halogen, amine, hydroxyl, or a combination thereof; R³ can be any substitution, including but not limited to O, alkyl, or a combination thereof; and each X can independently be O, N. Se, S, alkyl, or a combination thereof. In one embodiment, the linking agent includes one or more phosphorester bonds and one or more phosphoramide bonds, and can be represented by the formula:

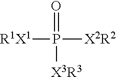

wherein X and X² are, independently, O, N, Se, S or alkyl; R¹ and R² are independently, one or more photoreactive groups, and X³ is O, N, Se, S, alkyl or aryl; R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or a hydroxyl or salt thereof. In one embodiment, the hydroxyl salt counterion is lithium, sodium, potassium, or a combination thereof.

In one embodiment, the linking agent comprises a triphosphorester, which can be represented by the formula.

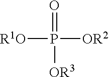

wherein R¹ and R² are independently, one or more photoreactive groups, and R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or hydrogen, or a hydroxyl salt. In one embodiment, the hydroxyl salt counterion is lithium, sodium, potassium, or a combination thereof.

Some specific embodiments include the following linking agents:
(a) bis(4-benzoylphenyl) hydrogen phosphate:

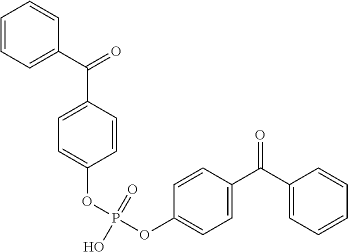 [SMILES: O=C(c1ccccc1)c1ccc(OP(=O)(O)Oc2ccc(C(=O)c3ccccc3)cc2)cc1]

(b) sodium bis(4-benzoylphenyl phosphate):

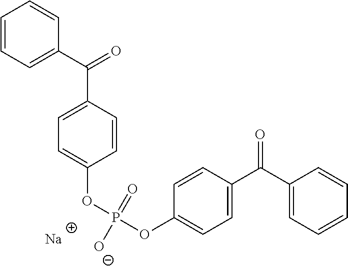 [SMILES: O=C(c1ccccc1)c1ccc(OP(=O)([O-])Oc2ccc(C(=O)c3ccccc3)cc2)cc1 ; [Na+]]

(c) tris(4-benzyolphenyl) phosphate):

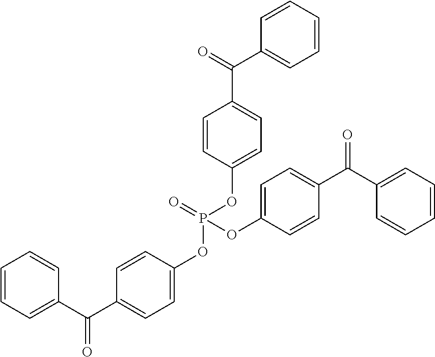 [SMILES: O=C(c1ccccc1)c1ccc(OP(=O)(Oc2ccc(C(=O)c3ccccc3)cc2)Oc2ccc(C(=O)c3ccccc3)cc2)cc1]

(d) tetrakis(4-benzoylphenyl)methylenebis(phosphonate)

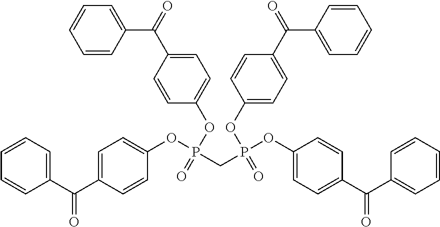 [SMILES: O=C(c1ccccc1)c1ccc(OP(=O)(CP(=O)(Oc2ccc(C(=O)c3ccccc3)cc2)Oc2ccc(C(=O)c3ccccc3)cc2)Oc2ccc(C(=O)c3ccccc3)cc2)cc1]

In another embodiment, the linking agent comprises a triphosphoramide, which can be represented by the formula.

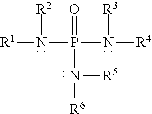

wherein R¹-R⁶ are independently, a photoreactive group, a hydroxyl or salt thereof, alkyl or aryl, or a combination thereof, wherein at least two of R¹-R⁶ are, independently, a photoreactive group. In one embodiment, the hydroxyl salt counterion is lithium, sodium, potassium, or a combination thereof In a more particular embodiment, R¹-R⁶ are independently cyclic, linear or branched, saturated or unsaturated, aromatic or heteroaromatic, or a combination thereof. In a more particular embodiment, R¹-R⁶ are, independently, phenyl, methyl, ethyl, isopropyl, t-butyl, or a combination thereof.

In some embodiments, the photoactivatable cross-linking agent can be ionic, and can have good solubility in an aqueous composition, such as the first and/or second coating composition. Thus, in some embodiments, at least one ionic photoactivatable cross-linking agent is used to form the coating. In some cases, an ionic photoactivatable cross-linking agent can crosslink the polymers within the second coating layer which can also improve the durability of the coating.

Any suitable ionic photoactivatable cross-linking agent can be used. In some embodiments, the ionic photoactivatable cross-linking agent is a compound of formula I: X₁-Y-X₂ where Y is a radical containing at least one acidic group, basic group, or a salt of an acidic group or basic group. X₁ and X₂ are each independently a radical containing a latent photoreactive group. The photoreactive groups can be the same as those described herein. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like. For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X₁ and X₂ can contain photoreactive groups that include aryl ketones. Such photoactivatable cross-linking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bis[2-(4-benzoylbenzyldimethylammonio)ethyl]-4-benzoylbenzylmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable cross-linking agent can be a compound having the formula:

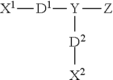

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable cross-linking agents are described in US Patent Application Publication US 2011/0144373 (Swan et al., “Water Soluble Degradable Crosslinker”), the disclosure of which is incorporated herein by reference.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable cross-linking agent can be represented by the formula:

PG²-LE²-X-LE¹-PG¹

wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable cross-linking agents can include, for example, those described in U.S. Provisional Application 61/494,724 filed Jun. 8, 2011 (now U.S. application Ser. No. 13/490,994) (Swan et al., “Photo-Vinyl Primers/Crosslinkers”), the disclosure of which is incorporated herein by reference. Exemplary cross-linking agents can include non-ionic photoactivatable cross-linking agents having the general formula R¹-X-R², wherein R¹ is a radical comprising a vinyl group, X is a radical comprising from about one to about twenty carbon atoms, and R² is a radical comprising a photoreactive group.

Some suitable cross-linking agents are those formed by a mixture of the chemical backbone molecule (such as pentaerythritol) and an excess of a derivative of the photoreactive group (such as 4-bromomethylbenzophenone). An exemplary product is tetrakis(4-benzoylbenzyl ether) of pentaerythritol (tetrakis(4-benzoylphenylmethoxymethyl)methane). See U.S. Pat. Nos. 5,414,075 and 5,637,460.

A single photoactivatable cross-linking agent or any combination of photoactivatable cross-linking agents can be used in forming the coating. In some embodiments, at least one nonionic cross-linking agent such as tetrakis(4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis(4-benzoylbenzyldimethylammonium) salt or at least one anionic photoactivatable cross-linking agent such as 4,5-bis(4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

An exemplary cross-linking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

A further exemplary cross-linking agent is ethylenebis (4-benzoylbenzyldimethylammonium) dibromide. This agent can be prepared as described in U.S. Pat. No. 5,714,360, the content of which is herein incorporated by reference.

Further cross-linking agents can include the cross-linking agents described in U.S. Publ. Pat. App. No. 2010/0274012 and U.S. Pat. No. 7,772,393 the content of all of which is herein incorporated by reference.

In some embodiments, cross-linking agents can include boron-containing linking agents including, but not limited to, the boron-containing linking agents disclosed in US 61/666,516, entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

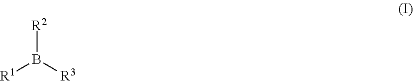

wherein R¹ is a radical comprising a photoreactive group; R² is selected from OH and a radical comprising a photoreactive group, an akyl group and an aryl group; and R³ is selected from OH and a radical comprising a photoreactive group. In some embodiments the bonds B-R¹, B-R² and B-R³ can be chosen independently to be interrupted by a heteroatom, such as O, N, S, or mixtures thereof.

Additional agents for use with embodiments herein can include stilbene-based reactive compounds including, but not limited to, those disclosed in US 61/736,436, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, cross-linking agents, hydrophilic coatings, and associated reagents are disclosed in US2011/0059874; US 2011/0046255; and US 2010/0198168, the content of all of which is herein incorporated by reference.

### Methods of Forming the Coating

In some embodiments, a first coating solution is formed by combining compounds with a solvent. For example, the compounds can include those described herein with respect to the barrier membrane. The solvent for the first coating solution can include various components depending on the specific components of the formulation. In some embodiments, the solvent can include or more of isopropyl alcohol (IPA), other alcohols, water, acetone, DMSO, or other solvents including organic or inorganic compounds.

In some embodiments, a second coating solution is formed by combining compounds with a solvent. For example, the compounds can include those described herein with respect to the non-fouling, tissue compatible coating.

The viscosity of the solutions can vary. In some embodiments, the viscosity of the second solution is less than about 100 centipoise (cP). In some embodiments, the viscosity of the second solution is equal to or less than about 90, 80, 70 60, 50, 40, 30, 20, or 10 cP.

The first coating solution can be applied to a substrate. Prior to application of the coating solution to the substrate, many different pretreatment steps can be taken. In some embodiments, the surface of the substrate can be cleaned. For example, the surface can be wiped or dipped into an alcohol such as isopropyl alcohol. In some embodiments, the substrate can be put into a detergent solution such as a VALTRON solution and sonicated. In some embodiments, a compound can be disposed on the surface of the substrate to act as a tie layer. In some embodiments the surface of the substrate can be sterilized.

Many different techniques can be used to apply the solution to the substrate. By way of example, exemplary techniques can include drop coating, blade coating, dip coating, spray coating, and the like. In various embodiments, the solution is applied by dip coating. The speed of dip coating can vary. For example, the substrate can be dipped into the first coating solution and then withdrawn at speeds between 0.01 and 10 cm/s. In some embodiments, the substrate can be dipped into the first coating solution and then withdrawn at speeds between 0.1 and 4 cm/s. In some embodiments, the substrate can be dipped into the first coating solution and then withdrawn at speeds between 0.1 and 0.5 cm/s. In some embodiments, the substrate can be withdrawn at speeds between 0.2 and 0.4 cm/s. In some embodiments, the substrate can be withdrawn at speeds of about 0.3 cm/s.

In some embodiments, after the first coating solution is applied to the substrate, then actinic radiation, such as UV radiation, can be applied to activate photoreactive groups within the components of the first coating solution forming the first layer. Actinic radiation can be provided by any suitable light source that promotes activation of the photoreactive groups. Preferred light sources (such as those available from Dymax Corp.) provide UV irradiation in the range of 190 nm to 360 nm. An exemplary UV light source is a Dymax 2000-EC series UV flood lamp with a 400 Watt metal halide bulb. A suitable dose of radiation is in the range of from about 0.5 mW/cm² to about 2.0 mW/cm². Optionally, the first coating solution can be dried, before or during application of the actinic radiation. However, in other embodiments, no actinic radiation is applied to the first coating solution. In some embodiments other curing steps are performed on the deposited first coating solution.

The second coating solution can be applied on top of the first coating layer. Many different techniques can be used to apply the solution to the substrate. In a particular embodiment, the solution is applied by dip coating. The speed of dip coating can vary. For example, the substrate can be dipped into the second coating solution and then withdrawn at speeds between 0.01 and 10 cm/s. In some embodiments, the substrate can be dipped into the second coating solution and then withdrawn at speeds between 0.1 and 4 cm/s. In some embodiments, the substrate can be dipped into the second coating solution and then withdrawn at speeds between 0.1 and 0.5 cm/s. In some embodiments, the substrate can be withdrawn at speeds between 0.2 and 0.4 cm/s. In some embodiments, the substrate can be withdrawn at speeds of about 0.3 cm/s.

In some embodiments, after the second coating solution is applied, then actinic radiation, such as UV radiation at a desirable wavelength, can be applied to activate photoreactive groups within the components of the second coating solution. Optionally, the second coating solution can be dried, before or during application of the actinic radiation. In some embodiments, no actinic radiation is applied to the deposited second coating solution.

### Substrates

Substrates can be partially or entirely fabricated from a metal, ceramic, glass, or the like, or a combination thereof. Substrates can include polymers such as polyurethanes and polyurethane copolymers, polyethylene, polyolefins, styrene-butadiene copolymers, polyisoprene, isobutylene-isoprene copolymers (butyl rubber), including halogenated butyl rubber, butadiene-styrene-acrylonitrile copolymers, silicone polymers, fluorosilicone polymers, polycarbonates, polyamides, polyesters, polyvinyl chloride, polyether-polyester copolymers, polyether-polyamide copolymers, and the like. The substrate can be made of a single material, or a combination of materials.

Substrate polymers can also include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

In some embodiments, the substrate includes a polymer selected from the group consisting of polyamide, polyimide, polyether block amide (PEBAX), polyether ether ketone (PEEK), high density polyethylene (HDPE), polyethylene, polyurethane, and polyethylene vinyl acetate.

Metals that can be used in medical articles include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35.

### Medical Devices

Various embodiments herein include a medical device. Embodiments herein specifically include coated medical devices. Further details about exemplary medical devices are provided as follows. However, it will be appreciated that this is merely provided by way of example and that further variations are contemplated herein.

In various embodiments, the medical device can be an implantable, partially implantable, chronically implantable, transitorily implantable, or a wearable device. The medical device can be one to provide therapy and/or sensing and monitoring features.

In various embodiments, the medical device can specifically include a sensor (such as for physiological analytes such as glucose). In various embodiments, the medical device can specifically be an implantable glucose sensor

The sensor can include a sensor interface (e.g., a portion of the sensor interfacing with tissues and/or fluids of the body.

The sensor can be an optical sensor, an electrochemical sensor, and an electrical potential sensor. In various embodiments, the sensor interface can include an electrochemical sensor interface, an optical sensor interface, an electrical sensor interface (such as an electrode), or the like.

Thromboresistant coatings herein can be applied on any blood-contacting device including both short indwelling or permanently implanted devices. In some embodiments, coatings herein can be applied with or without in combination with a drug-eluting stent. In some embodiments, coatings herein can be applied to any device provided in along with a procedure or device requiring single antiplatelet therapy (SAPT). In various embodiments, coatings herein can be provided on a flow diverter to treat an aneurysm, on adjunctive stenting for coiling in aneurysm, on or along with a device such as a drug-eluting stent in Intracranial Artery Disease (ICAD), or the like.

However, it will be appreciated that methods and materials of the invention can be utilized to coat virtually any medical device. In particular, the coatings are particularly useful for medical articles that can be inserted into and moved within the body and/or those that contact tissues and/or fluids of the body. Coatings herein can be applied on any tissue contacting device requiring non protein fouling or non-inflammatory tissue reaction.

Exemplary medical devices include vascular implants and grafts, surgical devices; synthetic prostheses; vascular prosthesis including endoprosthesis, stent-graft, and endovascular-stent combinations; small diameter grafts, abdominal aortic aneurysm grafts; wound dressings and wound management device; hemostatic barriers; mesh and hernia plugs; patches, including uterine bleeding patches, atrial septic defect (ASD) patches, patent foramen ovale (PFO) patches, ventricular septal defect (VSD) patches, and other generic cardiac patches; ASD, PFO, and VSD closures; percutaneous closure devices, mitral valve repair devices; left atrial appendage filters; valve annuloplasty devices, catheters; central venous access catheters, vascular access catheters, abscess drainage catheters, drug infusion catheters, parenteral feeding catheters, intravenous catheters (e.g., treated with antithrombotic agents), stroke therapy catheters, blood pressure and stent graft catheters; interventional cardiology devices including guide wires and leads (e.g. pacing, delivering electricity, defibrillation); anastomosis devices and anastomotic closures; aneurysm exclusion devices; biosensors, such as glucose sensors; cardiac sensors (and other sensors for analytical purposes); birth control devices; breast implants; infection control devices; membranes; tissue scaffolds; tissue-related materials; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff; sewing cuff; spinal and neurological devices; nerve regeneration conduits; neurological catheters; neuropatches;

orthopedic devices such as orthopedic joint implants, bone repair/augmentation devices, cartilage repair devices; urological devices and urethral devices such as urological implants, bladder devices, renal devices and hemodialysis devices, colostomy bag attachment devices; biliary drainage products.

### Photoreactive Groups

As used herein, the term “photoreactive group” refers to a functional group that is capable of responding to a specific applied external stimulus to undergo active specie generation and form a covalent bond with an adjacent chemical structure, which can be provided by the same or a different molecule. Photoreactive groups are those groups of atoms in a molecule that retain their covalent bonds unchanged under conditions of storage but that, upon activation by an external energy source, form one or more covalent bonds with other molecules. In one embodiment, the photoreactive groups can generate active species such as free radicals upon absorption of electromagnetic energy. Photoreactive groups can be chosen to be responsive to various portions of the electromagnetic spectrum, including, for example, the ultraviolet and visible portions of the spectrum. Photoreactive groups are described, for example, in U.S. Pat. No. 5,002,582, the disclosure of which is incorporated herein by reference.

In some embodiments, the photoreactive group includes a substituent capable of reacting with halogenated or triflated linking element. In a more particular embodiment, the photoreactive group contains a hydroxyl (—OH) or amine (—NR₂) substituent, wherein the amine substituent can be a primary amine or a secondary amine.

In some embodiments, the photoreactive group includes a photoreactive aryl ketone, such as acetophenone, benzophenone, anthraquinone, anthrone, and anthrone-like heterocycles (i.e., heterocyclic analogs of anthrone such as those having N, O, or S in the 10-position), or their substituted (e.g., ring substituted) derivatives. Examples of aryl ketones include heterocyclic derivatives of anthrone, including acridone, xanthone, and thioxanthone, and their ring substituted derivatives. One example includes thioxanthone, and its derivatives, having excitation energies greater than about 360 nm. In one embodiment, the photoreactive group is a functionalized benzophenone with an amine or hydroxyl substituent at positions 3 or 4 (i.e., 3- or 4-aminobenzophenone or 3- or 4-hydroxybenzophenone). As discussed above, the functionalized benzophenone can include a linker between the benzophenone photoreactive group and the amine or hydroxyl substituent. Examples of linkers include an amine, an ether, linear or branched C₁-C₁₀ alkyl, or a combination thereof.

The functional groups of such ketones are readily capable of undergoing the activation/inactivation/reactivation cycle described herein. Benzophenone is one example of a photoreactive moiety that is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source. Photoactivatible aryl ketones such as benzophenone and acetophenone are subject to multiple reactivation in water and may increase coating efficiency.

The azides constitute one class of photoreactive groups and include derivatives based on arylazides (C₆R₅N₃) such as phenyl azide and particularly 4-fluoro-3-nitrophenyl azide, acyl azides (—CO—N₃) such as benzoyl azide and p-methylbenzoyl azide, azido formates (—O—CO—N₃) such as ethyl azidoformate, phenyl azidoformate, sulfonyl azides (—SO₂—N₃) such as benzenesulfonyl azide, and phosphoryl azides (RO)₂PON₃ such as diphenyl phosphoryl azide and diethyl phosphoryl azide. Diazo compounds constitute another class of photoreactive groups and include derivatives of diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as t-butyl alpha diazoacetoacetate. Other photoreactive groups include the diazirines (—CHN₂) such as 3-trifluoromethyl-3-phenyldiazirine, and ketenes (—CH═C═O) such as ketene and diphenylketene.

Upon activation of the photoreactive groups, the linking agents are covalently bound to each other, to other molecules, or to a surface by covalent bonds through residues of the photoreactive groups. Exemplary photoreactive groups, and their residues upon activation, are shown as follows.

| Photoreactive | Group |
|---|---|
| aryl azides | amine (R—NH—R′) |
| acyl azides | amide (R—CO—NH—R′) |
| azidoformates | carbamate (R—O—CO—NH—R′) |
| sulfonyl azides | sulfonamide (R—SO₂—NH—R′) |
| phosphoryl azides | phosphoramide ((RO)₂PO—NH—R′) |
| diazoalkanes | new C—C bond |
| diazoketones | new C—C bond and ketone |
| diazoacetates | new C—C bond and ester |
| beta-keto-alpha-diazoacetates | new C—C bond and beta-ketoester |
| aliphatic azo | new C—C bond |
| diazirines | new C—C bond |
| ketenes | new C—C bond |
| photoactivated ketones | new C—C bond and alcohol |

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A thromboresistant implantable, partially implantable, or wearable medical device comprising:
a substrate; and
a non-fouling basecoat layer, the non-fouling basecoat layer comprising
a hydrophilic component; and
a hydrophobic component;
wherein the non-fouling basecoat layer is disposed over the substrate;
a lubricious topcoat layer, the lubricious topcoat layer comprising
a photo-reactive polyvinylpyrrolidone compound; and
a cross-linking agent; and
wherein the lubricious topcoat layer is disposed over the non-fouling basecoat layer.

2. (canceled)

3. The medical device of claim 1, the hydrophobic component comprising a poly(n-butyl methacrylate).

4. The medical device of claim 1, the hydrophilic component comprising a polyvinylpyrrolidone polymer.

5. The medical device of claim 4, the polyvinylpyrrolidone polymer comprising a cross-linked polyvinylpyrrolidone.

6. The medical device of claim 4, the hydrophilic component further comprising an acrylamide polymer, wherein the polyvinylpyrrolidone polymer and the acrylamide polymer are cross-linked.

7. (canceled)

8. The medical device of claim 1, wherein a ratio of the hydrophobic component to the hydrophilic component in the non-fouling basecoat layer is from about 2.5:1 to 3.5:1 by weight.

9. The medical device of claim 1, the non-fouling basecoat layer further comprising a heparin compound.

10. The medical device of claim 9, the heparin compound comprising a photoreactive group.

11-16. (canceled)

17. The medical device of claim 1, the lubricious topcoat layer further comprising a heparin compound.

18. The medical device of claim 17, the heparin compound comprising a photoreactive group.

19. The medical device of claim 17, wherein the heparin compound is cross-linked with the photo-reactive polyvinylpyrrolidone compound.

20. The medical device of claim 17, the heparin compound comprising an elutable heparin compound, wherein the elutable heparin compound is ionically complexed with a polycationic polymer.

21. The medical device of claim 20, the polycationic polymer comprising at least one selected from the group consisting of PEI, PHEMA-co-DMAEMA, and PEG-DMAEMA.

22. The medical device of claim 1, the lubricious topcoat layer further comprising a non-photoreactive polyvinylpyrrolidone.

23-27. (canceled)

28. The medical device of claim 8, the lubricious topcoat layer further comprising a polyacrylamide polymer.

29-31. (canceled)

32. The medical device of claim 8, the lubricious topcoat layer further comprising a polyzwitterion.

33-35. (canceled)

36. The medical device of claim 8, the lubricious topcoat layer further comprising an anionic polymer.

37. (canceled)

38. The medical device of claim 1, the lubricious topcoat layer further comprising:
a first layer; and
a second layer, wherein the second layer is disposed on the first layer and is different than the first layer.

39. (canceled)

40. The medical device of claim 1, the lubricious topcoat layer further comprising an elutable antiplatelet macromer.

41. (canceled)

42. The medical device of claim 1, the lubricious topcoat layer further comprising a non-prodrug anti-platelet agent.

43-81. (canceled)
